SEQUENCE LISTING (20 sequences)

SEQ ID NO: 1 - DNA, 2200 bp, Artificial Sequence
  Synthetic polynucleotide
gcccgggctcgagcccgggactagtaaaaattgaaaataaatacaaaggttcttgagggttgtgttaaattgaaagcgagaaataatcataaataatttcattatcgcgatatccgttaagtttgagctcccatgcggagccacgacgtgctgaccgtgcagttcctgatcctgggcatgctgctgatgacaggcggcgtgaccctggtccggaagaaccggtggctgctgctgaacgtgacctcagaggacctgggcaagaccttcagcgtgggcaccggcaactgcaccaccaacatcctggaagccaagtactggtgccccgacagcatggaatacaactgccccaacctgagccccagagaggaacccgacgacatcgactgctggtgctacggcgtggagaacgtgcgggtggcctacggcaagtgcgacagcgccggcagaagccggcggagcaggcgggccatcgacctgcccacccacgagaaccacggcctgaaaacccggcaggaaaagtggatgaccggccggatgggcgagcggcagctgcagaagatcgagcgctggttcgtcaggaaccccttcttcgccgtcaccgccctgaccatcgcctacctggtcggcagcaacatgacccagcgggtggtgatcgccctgctggtcctggccgtgggccctgcctacagcgcccactgcatcggcatcaccgaccgggacttcatcgagggcgtgcacggcggcacatgggtgtccgccaccctggaacaggacaagtgcgtgaccgtgatggcccccgataagcccagcctggacatcagcctggaaaccgtggccatcgacagacccgccgaagtgcggaaagtgtgctacaacgccgtgctgacccacgtgaagatcaacgacaagtgccccagcaccggcgaagcccacctggccgaggaaaacgagggcgacaacgcctgcaagcggacctactcagaccggggctggggcaacggctgcggcctgttcggcaagggcagcatcgtggcctgcgccaagttcacctgtgccaagagcatgagcctgttcgaggtggaccagaccaagatccagtacgtgatccgggcccagctgcacgtgggcgccaagcaggaaaactggaacaccgacatcaagaccctgaagttcgacgccctgagcggcagccaggaagtggagttcatcggctacggcaaggccacactggaatgccaggtgcagaccgccgtggacttcggcaacagctacatcgccgagatggaaaccgagagctggatcgtggaccggcagtgggcccaggacctgaccctgccctggcagagcggcagcggcggagtgtggcgggagatgcaccacctggtggagttcgagcccccccacgccgccaccatccgggtgctggccctgggtaaccaggaaggctccctgaaaacagccctgaccggcgccatgcgggtgaccaaggacaccaacgacaacaacctgtacaagctgcacggcgggcacgtgagctgccgggtcaagctgtctgccctgaccctgaagggcaccagctacaagatctgcaccgacaagatgttcttcgtgaagaaccccaccgacaccggccacggcaccgtggtgatgcaggtcaaagtgtctaagggcgctccctgccggatccccgtgatcgtggccgacgacctgacagccgccatcaacaagggcatcctggtgaccgtgaaccccatcgccagcaccaacgacgacgaggtgctgatcgaggtgaacccccccttcggcgactcctacatcatcgtgggcaggggcgacagccggctgacctaccagtggcacaaagagggcagcagcatcggcaagctgttcacccagaccatgaagggcgtggagcggctggccgtgatgggggacaccgcctgggacttcagcagcgctggcggcttcttcacctccgtgggcaagggcattcacaccgtgttcggcagcgccttccagggcctgtttggcggcctgaactggatcaccaaggtgatcatgggggccgtgctgatctgggtgggcatcaacacccggaacatgaccatgagcatgtcaatgatcctggtcggcgtgatcatgatgtttctgagcctgggcgtgggcgcctgagtcgactttttatgcggccgctacgtat

SEQ ID NO: 2 - protein, 679 aa, Artificial Sequence
  Synthetic Construct
MRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGAVLIWVGINTRNMTMSMSMILVGVIMMFLSLGVGA

SEQ ID NO: 3 - DNA, 2200 bp, Artificial Sequence
  Synthetic polynucleotide
agtatgttggggaaatatgaacctgacatgattaagattgctctttcggtggctggtagaagacgctagcactagtggatctaaaaattgaaattttattttttttttttggaatataaatagagctcccatgcggagccacgacgtgctgaccgtgcagttcctgatcctgggcatgctgctgatgacaggcggcgtgaccctggtccggaagaaccggtggctgctgctgaacgtgacctcagaggacctgggcaagaccttcagcgtgggcaccggcaactgcaccaccaacatcctggaagccaagtactggtgccccgacagcatggaatacaactgccccaacctgagccccagagaggaacccgacgacatcgactgctggtgctacggcgtggagaacgtgcgggtggcctacggcaagtgcgacagcgccggcagaagccggcggagcaggcgggccatcgacctgcccacccacgagaaccacggcctgaaaacccggcaggaaaagtggatgaccggccggatgggcgagcggcagctgcagaagatcgagcgctggttcgtcaggaaccccttcttcgccgtcaccgccctgaccatcgcctacctggtcggcagcaacatgacccagcgggtggtgatcgccctgctggtcctggccgtgggccctgcctacagcgcccactgcatcggcatcaccgaccgggacttcatcgagggcgtgcacggcggcacatgggtgtccgccaccctggaacaggacaagtgcgtgaccgtgatggcccccgataagcccagcctggacatcagcctggaaaccgtggccatcgacagacccgccgaagtgcggaaagtgtgctacaacgccgtgctgacccacgtgaagatcaacgacaagtgccccagcaccggcgaagcccacctggccgaggaaaacgagggcgacaacgcctgcaagcggacctactcagaccggggctggggcaacggctgcggcctgttcggcaagggcagcatcgtggcctgcgccaagttcacctgtgccaagagcatgagcctgttcgaggtggaccagaccaagatccagtacgtgatccgggcccagctgcacgtgggcgccaagcaggaaaactggaacaccgacatcaagaccctgaagttcgacgccctgagcggcagccaggaagtggagttcatcggctacggcaaggccacactggaatgccaggtgcagaccgccgtggacttcggcaacagctacatcgccgagatggaaaccgagagctggatcgtggaccggcagtgggcccaggacctgaccctgccctggcagagcggcagcggcggagtgtggcgggagatgcaccacctggtggagttcgagcccccccacgccgccaccatccgggtgctggccctgggtaaccaggaaggctccctgaaaacagccctgaccggcgccatgcgggtgaccaaggacaccaacgacaacaacctgtacaagctgcacggcgggcacgtgagctgccgggtcaagctgtctgccctgaccctgaagggcaccagctacaagatctgcaccgacaagatgttcttcgtgaagaaccccaccgacaccggccacggcaccgtggtgatgcaggtcaaagtgtctaagggcgctccctgccggatccccgtgatcgtggccgacgacctgacagccgccatcaacaagggcatcctggtgaccgtgaaccccatcgccagcaccaacgacgacgaggtgctgatcgaggtgaacccccccttcggcgactcctacatcatcgtgggcaggggcgacagccggctgacctaccagtggcacaaagagggcagcagcatcggcaagctgttcacccagaccatgaagggcgtggagcggctggccgtgatgggggacaccgcctgggacttcagcagcgctggcggcttcttcacctccgtgggcaagggcattcacaccgtgttcggcagcgccttccagggcctgtttggcggcctgaactggatcaccaaggtgatcatgggggccgtgctgatctgggtgggcatcaacacccggaacatgaccatgagcatgtcaatgatcctggtcggcgtgatcatgatgtttctgagcctgggcgtgggcgcctgagtcgactttttatgcggccgctttggaaag

SEQ ID NO: 4 - protein, 679 aa, Artificial Sequence; identical to SEQ ID NO: 2
  Synthetic Construct

SEQ ID NO: 5 - protein, 718 aa, Yellow fever virus
  MISC_FEATURE: Yellow fever virus Pasteur 17D-204 yellow fever vaccine
ITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGAVLIWVGINTRNMTMSMSMILVGVIMMFLSLGVGA

SEQ ID NO: 6 - protein, 718 aa, Yellow fever virus
  MISC_FEATURE: Yellow fever virus YFV17D-213
ITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWTTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGAVLIWVGINTRNMTMSMSMILVGVIMMFLSLGVGA

SEQ ID NO: 7 - protein, 718 aa, Yellow fever virus
  MISC_FEATURE: Yellow fever virus 17D-204-South Africa vaccine
ITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGITDRDFIEGVHGGTWVSATLEQDKCVTVMAPDKPSLDISLETVAIDRPAEVRKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGVVLIWVGINTRNMTMSMSMIMVGVIMMFLSLGVGA

SEQ ID NO: 8 - protein, 718 aa, Yellow fever virus
  MISC_FEATURE: Yellow fever virus vaccine strain 17DD
ITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGITDRDFIEGVHGGTWVSATLEQDKCVTVMAPDKPSLDISLETVAIDRPAEARKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTSIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVPKGAPCRIPVIVADDLTAAINKGILVTVNPIASTNDDEVLIEVNPPFGDSYIIVGRGDSRLTYQWHKEGSSIGKLFTQTMKGVERLAVMGDVAWDFSSAGGFFTSVGKGIHTVFGSAFQGLFGGLNWITKVIMGAVLIWVGINTRNMTMSMSMILVGVIMMFLSLGVGA

SEQ ID NO: 9 - protein, 718 aa, Yellow fever virus
  MISC_FEATURE: Yellow fever virus strain Asibi
ITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWLVRNPFFAVTALTIAYLVGSNMTQRVVIALLVLAVGPAYSAHCIGITDRDFIEGVHGGTWVSATLEQDKCVTVMAPDKPSLDISLETVAIDGPAEARKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEAEFTGYGKATLECQVQTAVDFGNSYIAEMEKESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKMCTDKMSFVKNPTDTGHGTVVMQVKVPKGAPCKIPVIVADDLTAAINKGILVTVNPIASTNDDEVLIEVNPPFGDSYIIVGTGDSRLTYQWHKEGSSIGKLFTQTMKGAERLAVMGDAAWDFSSAGGFFTSVGKGIHTVFGSAFQGLFGGLNWITKVIMGAVLIWVGINTRNMTMSMSMILVGVIMMFLSLGVGA

SEQ ID NO: 10 - protein, 718 aa, Yellow fever virus
  MISC_FEATURE: Yellow fever virus strain French viscerotropic virus
ITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWLVRNPFFAVTALTIAYLVGSNMTQRVVIALLVLAVGPAYSAHCIGITDRDFIEGVHGGTWVSATLEQDKCVTVMAPDKPSLDISLETVAIDGPAEARKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTDIKTLKFDALSGSQEAEFTGYGKATLECQVQTAVDFGNSYIAEMEKESWIVDRQWAQDLTLPWQSGSGGVWRGMHHLVEFEPPHAATIRVLALGNQEGSLKTALTGAMRVTKDTNDNNLYKLHGGHVSCRVKLSALTLKGTSYKMCTDKMSFVKNPTDTGHGTVVMQVKVPKGAPCRIPVIVADDLTAAINKGILVTVNPIASTNDDEVLIEVNPPFGDSYIIVGTGDSRLTYQWHKEGSSIGKLFTQTMKGVERLAVMGDAAWDFSSAGGFFTSVGKGIHTVFGSAFQGLFGGLNWITKVIMGAVLIWVGINTRNMTMSMSMILVGVIMMFLSLGVGA

SEQ ID NO: 11 - DNA, 37 bp, Artificial Sequence
  Synthetic oligonucleotide
gttaacagtttccggtgaatgtgtagatccagatagt

SEQ ID NO: 12 - DNA, 44 bp, Artificial Sequence
  Synthetic oligonucleotide
gaagacgctagcactagtgcggccgctttggaaagttttatagg

SEQ ID NO: 13 - DNA, 44 bp, Artificial Sequence
  Synthetic oligonucleotide
gcggccgcactagtgctagcgtcttctaccagccaccgaaagag

SEQ ID NO: 14 - DNA, 27 bp, Artificial Sequence
  Synthetic oligonucleotide
cgtacgttattatatccataggaaagg

SEQ ID NO: 15 - DNA, 55 bp, Artificial Sequence
  Synthetic oligonucleotide
ctagcgacaagcttgcaggatccactaggcctataactagtccgctcgagattgc

SEQ ID NO: 16 - DNA, 55 bp, Artificial Sequence
  Synthetic oligonucleotide
ggccgcaatctcgagcggactagttataggcctagtggatcctgcaagcttgtcg

SEQ ID NO: 17 - DNA, 35 bp, Artificial Sequence
  Synthetic oligonucleotide
cgccgtcgactatattagacaatactacaattaac

SEQ ID NO: 18 - DNA, 28 bp, Artificial Sequence
  Synthetic oligonucleotide
atatggatcctctaccagccaccgaaag

SEQ ID NO: 19 - DNA, 56 bp, Artificial Sequence
  Synthetic oligonucleotide
ctagtggatctaaaaattgaaattttattttttttttttggaatataaatagagct

SEQ ID NO: 20 - DNA, 48 bp, Artificial Sequence
  Synthetic oligonucleotide
ctatttatattccaaaaaaaaaaaataaaatttcaatttttagatcca

(19) United States
(12) Patent Application Publication
(10) Pub. No.: US 2012/0328649 A1
(43) Pub. Date: Dec. 27, 2012

(54) Recombinant Viral Vectors and Methods for Inducing an Immune Response to Yellow Fever Virus

(75) Inventors: Falko-Günter Falkner, Orth/Donau (AT); Birgit Schäfer, Wein (AT); Georg Holzer, Wien (AT); P. Noel Barrett, Klosterneuburg/Weidling (AT); Hartmut Ehrlich, Wien (AT)

(73) Assignees: BAXTER HEALTHCARE S.A., Glattpark (Opfikon) (CH); BAXTER INTERNATIONAL INC., Deerfield, IL (US)

(21) Appl. No.: 13/240,794

(22) Filed: Sep. 22, 2011

Related U.S. Application Data
(60) Provisional application No. 61/385,858, filed on Sep. 23, 2010.

(51) Int. Cl. A61K 39/295; A61P 37/04; C12N 7/01

(52) U.S. Cl. 424/199.1; 435/235.1

(57) ABSTRACT

The present invention relates to recombinant viral vectors and methods of using the recombinant viral vectors to induce an immune response to yellow fever virus. The invention provides recombinant viral vectors based on the non-replicating modified vaccinia virus Ankara or based on a D4R-defective vaccinia virus. When administered according to methods of the invention, the recombinant viral vectors induce a broad immune response to yellow fever virus and demonstrate an excellent safety profile.

This application claims the benefit of U.S. Provisional Patent Application No. 61/385,858 filed Sep. 23, 2010. The provisional application is hereby incorporated by reference in its entirety.

### FIELD OF THE INVENTION

The present invention relates to recombinant viral vectors and methods of using the recombinant viral vectors to induce an immune response to yellow fever virus. The invention provides recombinant viral vectors based on the non-replicating modified vaccinia virus Ankara or based on a D4R-defective vaccinia virus. When administered according to methods of the invention, the recombinant viral vectors induce a broad immune response to yellow fever virus and demonstrate an excellent safety profile.

### BACKGROUND OF THE INVENTION

Yellow fever (YF) still represents a constant threat to public health in endemic regions of tropical Africa and South America. The World Health Organization (WHO) estimated that 200,000 cases occur annually with 30,000 fatalities (WHO 2009). Yellow fever virus (YFV), a single-stranded RNA virus, belongs to the family of the Flaviviridae and is transmitted by mosquitoes (Lindenbach B D, Thiel H J, and Rice C M 2007). Yellow fever disease can be divided into three stages. After an incubation period of three to six days, patients develop febrile illness with symptoms like fever, malaise, lower back pain, headache, myalgia, nausea, vomiting, and prostration lasting three to four days. Symptoms disappear for two to forty-eight hours before fifteen to twenty-five percent of the patients enter the third phase, the period of intoxication, characterized by fever, vomiting, epigastric pain, hemorrhagic diathesis, jaundice, and liver and renal failure. Death occurs in twenty to fifty percent of severe YF cases on the seventh to tenth day (Monath 2001; Monath 2004; Gubler, Kuno, and Markoff 2007).

As early as 1937, a live attenuated vaccine strain, yellow fever 17D, was developed based on the Asibi wild-type strain by passage in mouse and chick tissue cultures (Theiler and Smith 1937; Stokes, Bauer, and Hudson 2001). The 17D vaccine has been in use for many decades and has been administered to more than 400 million people (Monath 2001).

The YFV envelope (E) protein plays a dominant role in the induction of a protective immune response. In animal studies, purified E protein or recombinant vaccinia virus expressing the precursor of the membrane and the envelope proteins (prME) induce high levels of neutralizing antibodies and confer immunity against lethal YFV infection (Brandriss, Schlesinger, and Walsh 1990; Pincus et al. 1992). Additionally, passive transfer of monoclonal anti-E antibodies demonstrated that the antibody mediated immunity was sufficient to protect mice (Brandriss et al. 1986). As an approach of an YFV vaccine that predominantly targets the humoral immune response, an inactivated whole virus candidate vaccine has recently been described (Monath et al. 2010). However, recent data point also to an important role for the cellular immune response. CD4 lymphocytes bearing a Th1 phenotype in combination with antibodies play a critical role in virus clearance (Liu and Chambers 2001). CD8 T cells that were induced by YFV-17D exhibited all characteristics necessary for protective cellular immunity, such as broad specificity, robust proliferation, high magnitude, and long term persistence (Miller et al. 2008; Akondy et al. 2009). A high number of CD8- and CD4-specific T cell epitopes were mapped in the envelope protein (Co et al. 2002; van der Most et al. 2002; Maciel, Jr. et al. 2008).

Recombinant vaccines based on modified vaccinia virus Ankara (MVA) have been used in many non-clinical and clinical studies (Cebere et al. 2006; Bejon et al. 2007; Brookes et al. 2008; Kaufman et al. 2009). MVA has proven to be exceptionally safe (Drexler, Staib, and Sutter 2004). No significant side effects have been obtained when MVA was administered to more than 120,000 human patients in the context of the smallpox eradication (Stickl et al. 1974; Mayr et al. 1978). Due to a block in virion morphogenesis the highly attenuated vaccinia virus strain fails to productively replicate in human and most other mammalian cells (Carroll and Moss 1997; Drexler et al. 1998; Wyatt et al. 1998). Nevertheless, the ability to express viral and foreign genes in the early and late stage is retained. These characteristics make MVA a promising live vaccine vector that induces humoral and cellular immune responses and exhibits a high safety profile. Another non-replicating vaccinia virus, the D4R-defective vaccinia virus (dVV), was generated by targeted deletion of the essential VV uracil DNA glycosylase gene (D4R) which is involved in viral DNA synthesis. Thus, in any wild-type cells, the replication cycle is blocked at the stage of viral genomic replication prior to late gene expression. For propagation of dVV, an engineered cell line is used that complements the deleted viral D4R function (Holzer and Falkner 1997; Mayrhofer et al. 2009). Due to this well-defined deletion the non-replicating virus dVV represents a safe vaccine vector (Ober et al. 2002).

U.S. Pat. Nos. 6,998,252; 7,015,024; 7,045,136 and 7,045,313 relate to recombinant poxviruses, such as vaccinia.

MVA-based vaccines have been used in clinical studies, for instance, against HIV(Cebere et al. 2006), tuberculosis (Brookes et al. 2008), malaria (Bejon et al. 2007) and cancer (Kaufman et al. 2009). In all of these studies, at least two doses were used. The human dose of an MVA-based vaccine was 5×10⁷ to 5×10⁸ PFU as applied in clinical trials (Cebere et al. 2006; Tykodi and Thompson 2008; Brookes et al. 2008).

U.S. Pat. Nos. 5,514,375 and 5,744,140 relate to recombinant poxvirus such as a host range mutant of vaccinia virus containing foreign DNA from flavivirus such as YFV. U.S. Pat. No. 5,021,347 relates to a recombinant vaccinia virus such as an attenuated smallpox virus having Japanese encephalitis virus E-protein cDNA inserted into a non-essential region. U.S. Pat. No. 5,766,882 relates to defective, recombinant poxvirus lacking an essential function containing a foreign DNA. Holzer et al. 1999 (Holzer et al. 1999) describes a uracil DNA glycoylase-deficient vaccinia virus vector carrying the tick-borne encephalitis virus prM/E gene.

In a previous study (Pincus et al. 1992), a single dose of 1×10⁷ PFU of the replication competent vaccinia virus Western Reserve strain expressing the YFV-17D prME could only partially protect mice against a 100-fold LD₅₀ challenge with the French neurotropic YFV strain. Even after two inoculations, only 94% of the animals survived. In studies with recombinant MVA expressing the Japanese encephalitis virus (JEV) prME genes three doses of 2×10⁶ infectious units were necessary to protect mice against JEV challenge with 10⁵ LD₅₀ (Nam et al. 1999).

The 17D vaccine, formerly classified as one of the most effective and safest available (Barrett 1997) is now considered to be less safe (Monath 2007). Recent studies revealed a number of vaccine related serious adverse events. Per 100,000 vaccinations 0.8 subjects developed vaccine-associated neurotropic disease (Lindsey et al. 2008) and 1 in 200,000 to 400,000 vaccinees developed viscerotropic disease (Monath 2007). Within the major traveler group, i.e. people over 60 years of age, the incidence rate rises to 1 for every 50,000 vaccinations. Additionally, serious adverse outcomes, including death, have been reported in Spain, Brazil, United States, Australia, and Thailand across all age groups (Vasconcelos et al. 2001; Martin et al. 2001; Chan et al. 2001; Kengsakul, Sathirapongsasuti, and Punyagupta 2002; Gerasimon and Lowry 2005; Doblas et al. 2006; Belsher et al. 2007). Thus, there is a need in the art for a vaccine with an improved safety profile.

### DETAILED DESCRIPTION

The present invention provides recombinant viruses (also referred to as recombinant viral vectors herein) useful for generating an immune response to YFV. The recombinant viruses are based on the non-replicating vaccinia viruses, MVA and dVV, and encode a YFV prME polypeptide. When administered, the recombinant viruses induce YFV specific humoral and cellular immune responses (including a CD8 and CD4 T cell response) at levels similar to the 17D vaccine and protect mice against a lethal YFV challenge even subsequent to pre-immunization with wild-type vaccinia virus. In addition, the recombinant viruses exhibit an improved safety profile in mice compared to the 17D vaccine. The recombinant viruses are therefore contemplated to be useful as vaccines in humans.

The prME amino acid sequence encoded by an open reading frame in recombinant viruses of the invention may be, for example, the YFV-17D prME amino acid sequence set out in SEQ ID NO: 2; the Pasteur 17D-204 YFV vaccine prME polypeptide sequence set out in SEQ ID NO: 5 (from NCBI Genbank CAB37419.1); the YFV YFV17D-213 prME polypeptide sequence set out in SEQ ID NO: 6 (from NCBI Genbank AAC54268.1), the South Africa 17D-204 YFV vaccine prME polypeptide sequence set out in SEQ ID NO: 7 (from NCBI Genbank AAC35907.1), the YFV vaccine strain 17DD prME polypeptide sequence set out in SEQ ID NO: 8 (from NCBI Genbank AAC54267.1), the YFV strain Asibi prME polypeptide sequence set out in SEQ ID NO: 9 (from NCBI Genbank AAT58050) or the French viscerotropic YFV strain prME polypeptide sequence set out in SEQ ID NO: 10 (from NCBI Genbank AAA99713.1). In some embodiments, the prME polypeptide encoded by an open reading frame in a recombinant virus of the invention may vary in sequence from SEQ ID NO: 2, 5, 6, 7, 8, 9 or 10 but the prME polypeptide retains the ability to induce a protective immune response when the recombinant virus is administered to an individual. In these embodiments, the prME polypeptide may be about 80%, about 85%, about 90%, about 91%, about 92%, about 93%, about 94%, about 95%, about 95%, about 97%, about 98% or about 99% identical to SEQ ID NO: 2, 5, 6, 7, 8, 9 or 10.

In recombinant viruses of the invention, the open reading frame encoding the YFV prME polypeptide may be codon-optimized for expression in human cells. In these embodiments, one or more (or all) of the codons in the naturally occurring YFV prME open reading frame have been replaced in the codon-optimized YFV prME open reading frame with codons frequently used in genes in human cells (sometimes referred to as preferred codons). Codon-optimization, in general, has been used in the field of recombinant gene expression to enhance expression of polypeptides in cells.

Gene cassettes encoding YFV prME polypeptides in recombinant viruses of the invention include an YFV prME open reading frame under the control of (i.e., operatively linked to) a promoter that functions (i.e., directs transcription of the open reading frame) in the recombinant vaccinia virus. In execmplary embodiments, expression of prME polypeptide from gene cassettes is under the control of the strong early/late vaccinia virus mH5 promoter or the synthetic early/late selP promoter (Chakrabarti, Sisler, and Moss 1997).

In one aspect, the invention provides recombinant, modified vaccinia virus Ankara (MVA) comprising a YFV prME gene cassette. In one exemplified embodiment, the gene cassette comprises the strong early/late vaccinia virus promoter mH5 operatively linked to a human codon-optimized YFV-17D prME open reading frame and a vaccinia virus early transcription stop signal as set out in SEQ ID NO: 1. The prME amino acid sequence encoded by the open reading frame is set out in SEQ ID NO: 2 (and SEQ ID NO: 4). The codon-optimized sequence of the prME open reading frame corresponds to the nucleotides 419-2452 of the YFV-17D vaccine strain genome (Accession number NC_{—}002031). In another exemplified embodiment, the gene cassette as set out in SEQ ID NO: 3 comprises a synthetic early/late promoter (selP) (Chakrabarti, Sisler, and Moss 1997) operatively linked to the same human codon-optimized prME open reading frame. In other embodiments, the open reading frame encoding the prME polypeptide may be any human codon-optimized open reading frame encoding the YFV-17D prME amino acid sequence set out in SEQ ID NO: 2.

In yet other embodiments, the recombinant MVA YFV prME gene cassette may encode the Pasteur 17D-204 YFV vaccine prME polypeptide sequence set out in SEQ ID NO: 5 (from NCBI Genbank CAB37419.1); the YFV YFV17D-213 prME polypeptide sequence set out in SEQ ID NO: 6 (from NCBI Genbank AAC54268.1), the South Africa 17D-204 YFV vaccine prME polypeptide sequence set out in SEQ ID NO: 7 (from NCBI Genbank AAC35907.1), the YFV vaccine strain 17DD prME polypeptide sequence set out in SEQ ID NO: 8 (from NCBI Genbank AAC54267.1), the YFV strain Asibi prME polypeptide sequence set out in SEQ ID NO: 9 (from NCBI Genbank AAT58050) or the French viscerotropic YFV strain prME polypeptide sequence set out in SEQ ID NO: 10 (from NCBI Genbank AAA99713.1). The polypeptide sequences may vary as discussed in paragraph [0012] above. The open reading frames encoding these prME polypeptide sequences may also be human codon-optimized sequences. Expression of the gene cassettes may be under the control of, for example, the strong early/late vaccinia virus mH5 promoter or the synthetic early/late selP promoter.

The prME gene cassette may be inserted in the MVA in non-essential regions of the genome, such as the deletion I region, the deletion II region, the deletion III region, the deletion IV region, the thymidine kinase locus, the D4/5 intergenic region, or the HA locus. In an exemplified embodiment, the insertion is in the deletion III region. The recombinant MVA is derived from an MVA free of bovine spongiform encephalopathy (BSE) such as MVA74 LVD6 obtained from the National Institutes of Health.

The recombinant MVA expressing a YFV prME gene cassette may be formulated as a pharmaceutical composition according to standard methods in the art. In some embodiments, the pharmaceutical composition comprises a pharmaceutically acceptable carrier. The term “pharmaceutically acceptable carrier” includes any and all clinically useful solvents, dispersion media, coatings, antibacterial and antifungal agents, isotonic and absorption delaying agents, buffers, and excipients, such as a phosphate buffered saline solution, 5% aqueous solution of dextrose or mannitol, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers useful for the composition depend upon the intended mode of administration of the active agent.

The invention provides methods of inducing an immune response to YFV in an individual comprising administering the pharmaceutical composition to the individual. In the methods, the pharmaceutical composition may be administered as a single dose, a double dose or multiple doses. As exemplified herein, a single shot of 10⁵ TCID₅₀ of recombinant MVA induced comparable protection in mice to the single dose (10⁴ TCID₅₀) of YFV-17D. The immunization route in humans may be intramuscular (i.m.) or subcutaneous (s.c.). The range of the human immunization dose may be about 10⁶ to about 10⁹ PFU. The methods of the invention induce humoral and cellular immune responses in the individual. Moreover, in embodiments of the invention the methods induce a protective immune response in the individual. The protective immune response may be where subsequent exposure of the individual to YFV does not result in febrile illness. Febrile illness includes symptoms such as fever, malaise, lower back pain, headache, myalgia, nausea, vomiting, and prostration. The protective immune response may be where subsequent exposure to YFV does not result in a third phase of infection characterized, for example, by fever, vomiting, epigastric pain, hemorrhagic diathesis, jaundice, and liver and renal failure. The protective immune response may be where subsequent exposure to YFV does not result in fatal infection.

Also provided are methods of producing a recombinant MVA expressing a YFV prME gene cassette comprising the steps of: a) infecting primary chicken embryo cells or a permanent avian cell line with MVA, b) transfecting the infected cells with a plasmid comprising the prME gene cassette and comprising DNA flanking the gene cassette that is homologous to a non-essential region of the MVA genome, c) growing the cells to allow the plasmid to recombine with the MVA genome during replication of the MVA in chicken cells thereby inserting the prME gene cassette into the MVA genome in the non-essential region, and d) obtaining the recombinant MVA produced. Exemplary chicken embryo cells are described in U.S. Pat. No. 5,391,491. (Slavik, Ciampor, and Mayer 1983) Other avian cells (e.g., DF-1) are also contemplated. In the methods, the non-essential MVA region is the deletion I region, the deletion II region (Meyer, Sutter, and Mayr 1991), the deletion III region (Antoine et al. 1996), the deletion IV region (Meyer, Sutter, and Mayr 1991) (Antoine et al. 1998), the thymidine kinase locus (Mackett, Smith, and Moss 1982), the D4/5 intergenic region (Holzer et al. 1998), or the HA locus (Antoine et al. 1996). In one exemplified embodiment, the insertion is in the deletion III region. Genes could additionally inserted into any other suitable genomic region or intergenomic recgions.

In another aspect, recombinant, D4R-defective vaccinia viruses (dVV) expressing a YFV prME gene cassette are provided. In one exemplified embodiment, the gene cassette comprises the strong early/late vaccinia virus promoter mH5 operatively linked to a human codon-optimized YFV-17D prME open reading frame and a vaccinia virus early transcription stop signal as set out in SEQ ID NO: 1. The prME amino acid sequence encoded by the open reading frame is set out in SEQ ID NO: 2 (and SEQ ID NO: 4). The sequence of the prME open reading frame corresponds to the nucleotides 419-2452 of the YFV-17D vaccine strain genome (Accession number NC_{—}002031). In another embodiment, the gene cassette as set out in SEQ ID NO: 3 comprises a synthetic early/late promoter (selP) (Chakrabarti, Sisler, and Moss 1997) operatively linked to the same human codon-optimized prME open reading frame. The prME gene cassette may replace the D4R gene in a replicating vaccinia virus (VV) or may be inserted in a non-essential region of a dVV. In other embodiments, the open reading frame encoding the prME polypeptide in the recombinant dVV may be any human codon-optimized open reading frame encoding the YFV-17D prME amino acid sequence set out in SEQ ID NO: 2.

In yet other embodiments, the recombinant dVV YFV prME gene cassette may encode the Pasteur 17D-204 YFV vaccine prME polypeptide sequence set out in SEQ ID NO: 5 (from NCBI Genbank CAB37419.1); the YFV YFV17D-213 prME polypeptide sequence set out in SEQ ID NO: 6 (from NCBI Genbank AAC54268.1), the South Africa 17D-204 YFV vaccine prME polypeptide sequence set out in SEQ ID NO: 7 (from NCBI Genbank AAC35907.1), the YFV vaccine strain 17DD prME polypeptide sequence set out in SEQ ID NO: 8 (from NCBI Genbank AAC54267.1), the YFV strain Asibi prME polypeptide sequence set out in SEQ ID NO: 9 (from NCBI Genbank AAT58050) or the French viscerotropic YFV strain prME polypeptide sequence set out in SEQ ID NO: 10 (from NCBI Genbank AAA99713.1). The polypeptide sequences may vary as discussed in paragraph [0012] above. The open reading frames encoding these prME polypeptide sequences may also be human codon-optimized sequences. Expression of the gene cassettes may be under the control of, for example, the strong early/late vaccinia virus mH5 promoter or the synthetic early/late selP promoter.

The recombinant dVV expressing a YFV prME gene cassette may be formulated as a pharmaceutical composition. In some embodiments, the pharmaceutical composition comprises a pharmaceutically acceptable carrier. The term “pharmaceutically acceptable carrier” includes any and all clinically useful solvents, dispersion media, coatings, antibacterial and antifungal agents, isotonic and absorption delaying agents, buffers, and excipients, such as a phosphate buffered saline solution, 5% aqueous solution of dextrose or mannitol, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers useful for the composition depend upon the intended mode of administration.

The invention provides methods of inducing an immune response to YFV in an individual comprising administering the pharmaceutical composition to the individual. In the methods, the pharmaceutical composition may be administered as a single dose, a double dose or multiple doses. The immunization route in humans could be i.m. or s.c. The range of the immunization dose may be about 10⁶ to about 10⁹ PFU. The methods of the invention induce humoral and cellular immune responses in the individual. Moreover, in embodiments of the invention the methods induce a protective immune response in the individual. The protective immune response may be where subsequent exposure of the individual to YFV does not result in febrile illness. Febrile illness includes symptoms such as fever, malaise, lower back pain, headache, myalgia, nausea, vomiting, and prostration. The protective immune response may be where subsequent exposure to YFV does not result in a third phase of infection characterized, for example, by fever, vomiting, epigastric pain, hemorrhagic diathesis, jaundice, and liver and renal failure. The protective immune response may be where subsequent exposure to YFV does not result in fatal infection.

Also provided are methods of producing a recombinant dVV expressing a YFV prME comprising the steps of: a) infecting a D4R-complementing cell line with wild type VV (such as strain Lister/Elstree), b) transfecting the infected cells with a plasmid comprising a prME gene cassette and comprising DNA flanking the gene cassette that is homologous to the D3R and D5R regions of the wild type VV genome, c) growing the cells to allow the plasmid to recombine with the viral genome during replication of the viral genome in the D4R complementing cell line thereby inserting the prME gene cassette into viral genome between the D3R and D5R regions, and d) obtaining the recombinant dVV produced. The D4R-complementing cell line may be the rabbit RK44.20 cell line (Holzer and Falkner 1997), the African green monkey cVero-22 cell line (Mayrhofer et al. 2009) or any other cell line permissive for VV that provides the VV D4R gene product in trans.

In comparison to the 17D vaccine, the recombinant vaccinia viruses of the invention avoid contraindication in immunocompromised individuals, and cannot induce neurotropic and viscerotropic YFV vaccine associated adverse events because they are not replication competent in humans.

### FIGURES

FIG. 1 shows plasmid transfer vectors (i) and genome structures (ii) of MVA-YF (Aii) and dVV-YF (Bii). The plasmid vector pd3-lacZ-mH5-YFprMEco (Ai) targets the deletion III insertion site in the MVA genome. To obtain recombinant virus (Aii) without any auxiliary sequences, the transient lacZ/gpt screening marker is flanked by a 220 bp self repeat (R) of one of the MVA flanks that mediates removal of the marker cassette by spontaneous recombination. The insertion site for the plasmid vector pDW-mH5-YFprMEco (Bi) is the region between the ORFs D3R and D5R in the wild-type Lister/Elstree virus. The lacZ/gpt marker cassette is located between tandem DNA repeats (R) to achieve eventual removal of the marker cassette. The resulting recombinant defective virus (Bii) lacks the uracil DNA glycosylase gene (D4R), and still contains one tandem repeat. Both plasmids (Ai and Bi) contain the human codon optimized YFV prM and E coding region under the control of the early/late vaccinia virus mH5 promoter.

FIG. 2 shows double immunostaining of infected chicken cells (DF-1). (A) MVA-YF, (B) wild-type MVA and (C) MVA-YF/MVA spike control. After 4 days infected cells were fixed, incubated with guinea pig anti YFV-17D antiserum and anti-guinea pig IgG conjugated to peroxidase. Expressors of prME were visualized as black plaques staining with DAB solution with nickel. To detect MVA without prME expression, cells were incubated with rabbit anti vaccinia virus serum and anti-rabbit peroxidase conjugated IgG antibody and subsequent staining with DAB solution without nickel, resulting in brown plaques (prME non-expressors).

FIG. 3 shows YFV prME protein expression under permissive conditions. (A) Western blot of lysates from chicken cells (DF-1) infected with MVA-YF or the corresponding controls. MVA-YF (Lane 1), negative control, wild-type MVA (Lane 2), non-infected DF-1 cells (Lane 3), positive control YFV-17D infected DF-1 cells (17D, Lane 4), YFV-17D prepared from infected HeLa cells (17D control, Lane 5). (B) Western blot of lysates from cVero22 cells infected with dVV-YF or the corresponding controls. dVV-YF (Lane 1), negative control, wild-type dVV (Lane 2), non-infected cVero22 cells (Lane 3), positive control YFV-17D infected cVero22 (17D, Lane 4), 17D control (Lane 5). The band around 50 kDa represents the YFV envelope protein.

FIG. 4 shows a comparison of YFV prME protein expression levels under non-permissive conditions. (A) Western blot of (A) mouse muscle cells (Sol8) or (B) human cells (HeLa) infected with the recombinants or the corresponding controls. MVA-YF (Lane 1), dVV-YF (Lane 2), negative control wild-type MVA (Lane 3), negative control wild-type dVV (Lane 4), non-infected (A) Sol8 (B) HeLa cells (Lane 5), positive control respective cell line infected with YFV-17D (17D, Lane 6), 17D control (Lane 7). The band around 50 kDa represents the YFV envelope protein.

FIG. 5 shows protection studies in Balb/c mice. Animals were vaccinated i.m. in a single dose scheme with the indicated doses of (A) MVA-YF, (B) dVV-YF or with (C) the positive control YFV-17D (17D) and the negative controls wild-type MVA, defective vaccinia virus (dVV) or buffer (PBS). Challenge was i.c. 21 days later with 1×10⁵ TCID₅₀ of YFV-17D vaccine strain and monitored for 14 days. Results are the average of 3 individual experiments.

FIG. 6 shows cellular immune responses elicited against YFV E-antigen. (A) FACS analysis of the number of IFN-γ secreting CD4+ T-cells after two immunizations with MVA-YF, dVV-YF or the corresponding YFV-17D (17D) positive or wild-type MVA and dVV negative control. Splenocytes from mice were stimulated with 15 mer peptides of the YFV E-protein, E57-71 (E4; black bars), E129-143 (E5; grey bars) and E133-147 (E6; white bars). (B) FACS analysis of the number of IFN-γ secreting CD8⁺ T cells after the two immunizations as indicated above. Splenocytes from mice were stimulated with 9 mer peptides of the YFV E-protein, E60-68 (E1; black bars), E330-338 (E2; grey bars), E332-340 (E3 white bars). (C) FACS analysis of cytotoxic killing of peptide-pulsed target cells by specific CD8⁺ T cells. Target cells were loaded with 9 mer peptides of the YFV E-protein, E60-68 (E1; black bars), E330-338 (E2; grey bars), E332-340 (E3 white bars). The data are mean values (+/−SD) of two independent experiments.

FIG. 7 shows the results of experiments investigating the influence of pre-existing anti-vaccinia immunity on protection. Balb/c mice were i.m. vaccinated with wild-type vaccinia viruses and immunized 3 months later in a prime or prime/boost scheme with a suboptimal (1×10³ TCID₅₀) or optimal (1×10⁵ TCID₅₀) i.m. dose of MVA-YF or with 1×10⁴ TCID₅₀ of YFV-17D virus or buffer as controls. All animals were challenged i.c. with 1×10⁵ TCID₅₀ of YFV-17D and monitored for 14 days for survival. Immunization schemes and results appear in Table 2.

FIG. 8 shows the safety of recombinant candidate vaccines in BALB/c mice. (A) Animals were injected i.c. with 1×10⁵ to 1×10⁷ TCID₅₀ (only 1×10⁷ TCID₅₀ dose shown) of MVA-YF (bright grey line), dVV-YF (grey line) and the corresponding controls wild-type MVA (dotted line) and dVV (black line) and monitored for 21 days. (B) Mice were injected i.c. with YFV-17D vaccine at doses of 1×10¹ (bright grey line), 1×10² (grey line) or 1×10³ (dotted line), and monitored for 21 days.

### EXAMPLES

The present invention is illustrated by the following examples wherein Examples 1 and 2 respectively describe various embodiments of the insertion of a codon-optimized gene encoding the precursor of the membrane and envelope (prME) protein of the YFV strain into the non-replicating modified vaccinia virus Ankara and into the D4R-defective vaccinia virus. Examples 3 and 4 demonstrate the expression of the gene cassette from the recombinant viruses in various cells. The recombinant viruses were assessed for immunogenicity and protection in a mouse model and compared to the commercial YFV-17D vaccine in experiments described in Examples 5 and 6. The recombinant live viruses conferred full protection against lethal challenge already after a single low immunization dose of 10⁵ TCID₅₀, and induced substantial amounts of gamma interferon-secreting CD4- and functionally active CD8 T cells. Example 7 demonstrates that a pre-existing immunity against wild-type vaccinia virus had no negative influence on the protection. Example 8 shows that, unlike the classical 17D vaccine, none of the recombinant viruses caused morbidity or mortality following intracerebral administration to the mouse, demonstrating high safety profiles.

### Example 1

### Construction and Characterization of the Recombinant Virus MVA-YF

A recombinant MVA that expresses the prME coding sequence (CDS) of yellow fever strain 17D was constructed, and was termed MVA-YF. The prME CDS under the control of the vaccinia virus early/late mH5 promotor (Wyatt et al. 1996) was chemically synthesized. This allowed the removal of poxvirus early transcription termination signals (5TNT) present in the original sequence and the optimization of the open reading frame for human codon usage to achieve high expression levels in humans without modifying the amino acid sequence. The sequence of the gene cassette including the mH5 promoter is set out in SEQ ID NO: 1.

To generate MVA-YF, the codon-optimized (co) expression cassette was inserted into the newly constructed transfer plasmid pd3-lacZ-gpt resulting in the plasmid pd3-lacZ-mH5-YFprMEco (FIG. 1 Ai). This plasmid directs the foreign gene into the deletion III (delIII) region of MVA by homologous recombination. The transfer plasmid for recombination into the del III region of the MVA genome (FIG. 1 Aii), was constructed in the following steps (i)-(v).

(i) pd3-Script Pre1.

The left and right flanks of the del III region were amplified by PCR from genomic DNA of wild-type MVA by using the oligonucleotides oYF-8 (5′-GTT AAC AGT TTC CGG TGA ATG TGT AGA TCC AGA TAG T-3′) (SEQ ID NO: 11) and oYF-9 (5′-GAA GAC GCT AGC ACT AGT GCG GCC GCT TTG GAA AGT TTT ATA GG-3′) (SEQ ID NO: 12) for the right flank, and oYF-10 (5′-GCG GCC GCA CTA GTG CTA GCG TCT TCT ACC AGC CAC CGA AAG AG-3′) (SEQ ID NO: 13) and oYF-11 (5′-CGT ACG TTA TTA TAT CCA TAG GAA AGG-3′) (SEQ ID NO: 14) for the left flank. An overlapping PCR was performed with these two fragments as templates and the primers oYF-11 and oYF-8. The fragment was cloned into the vector pPCR-Script Amp SK (+) (Stratagene) resulting in the plasmid pd3-Script Pre1.

(ii) pd3-dlacZ/Notr-MCS.

The residual lacZ sequences and the NotI restriction site at Pos. 1617 of the pPCR-Script-Amp SK (+) plasmid were removed by BamHI, BsmFI or by BsiWI, Eel136II and mung bean digestion followed by blunt end religation resulting in the plasmid pd3-dlacZ/Notr. In order to introduce a multiple cloning site (NheI, HindIII, A1uI, BamHI, StuI, SpeI, XhoI, NotI) between the vaccinia DNA segments, the plasmid was cut with NheI and NotI, and a linker consisting of the annealed oligonucleotides oYF-50 (5′-CTA GCG ACA AGC TTG CAG GAT CCA CTA GGC CTA TAA CTA GTC CGC TCG AGA TTG C-3′) (SEQ ID NO: 15) and oYF-51 (5′ GGC CGC AAT CTC GAG CGG ACT AGT TAT AGG CCT AGT GGA TCC TGC AAG CTT GTC G-3′) (SEQ ID NO: 16) was inserted, resulting in pd3-dlacZ/Notr-MCS.

(iii) pDW2-repeat-delIII.

A delIII self repeat (R) of the left MVA flank (Staib et al. 2000) was generated to facilitate removal of lacZ/gpt gene cassette by internal homologous recombination during plaque purification. The delIII self repeat (220 bp) was amplified by PCR from pd3-Script using the oligonucleotides oYF-48 (5′-CGC CGT CGA CTA TAT TAG ACA ATA CTA CAA TTA AC-3′) (SEQ ID NO: 17) and oYF-49 (5′-ATA TGG ATC CTC TAC CAG CCA CCG AAA G-3′) (SEQ ID NO: 18) and cloned between the SalI and BamHI sites of pDW2 (Holzer et al. 1998) downstream of the gpt/lacZ gene cassette.

(iv) pd3-lacZ-gpt.

The lacZ/gpt delIII self repeat fragment of pDW2-repeat-delIII was cloned into pd3-lacZ/Notr-MCS using the HindIII and BamHI restriction sites, resulting to pd3-lacZ-gpt.

(v) pd3-lacZ-mH5-YFprMEco.

The open reading frame encoding the YFV prME (YFprMEco) gene (Accession Number NC_{—}002031, (Rice et al. 1985) under the control of the strong early/late vaccinia virus promoter mH5 was optimized for human codon usage (co) and synthesized (Geneart, Regensburg, Germany). The synthetic sequence is devoid of vaccinia virus early transcription stop signals; such signal was introduced immediately downstream of the coding region. The expression cassette was inserted into the SpeI/NotI site of pd3-lacZ-gpt resulting in pd3-lacZ-mH5-YFprMEco (FIG. 1 Ai).

Construction and purification of recombinant MVA-YF was carried out as follows (FIG. 1A (II)). Twenty micrograms of pd3-lacZ-mH5-YFprMEco plasmid were transfected into MVA-infected primary chicken embryo cells (CEC) by calcium phosphate precipitation. CEC has been generated from 12-day old chicken embryos and grown in Medium 199 (Gibco) containing 5% fetal calf serum (FCS), 100 UI/ml Pen/Strep (Lonza) and 100 UI/ml NEAA (Lonza). Recombinant virus was selected using the transient marker stabilization method as described previously (Scheiflinger, Dorner, and Falkner 1998). A purified MVA-YF clone was expanded for large scale propagation in CEC. After several rounds of plaque purification, initially with, then without, selective pressure (Wyatt et al. 1996) the final recombinant virus designated MVA-YF virus was obtained (FIG. 1 Aii). This virus contains the prME gene regulated by the vaccinia virus mH5 promotor in the MVA del III insertion site and is free of additional foreign sequences.

As an alternative, a plasmid equivalent to pd3-lacZ-mH5-YFprMEco but containing, between the SacI/SpeI restrictions sites, the synthetic E/L promoter (selP) (Chakrabarti et al. 1997) instead of the mH5 promoter was constructed. The selP promoter was generated by annealing the oligonucleotides oYF-39 (5′-CTA GTG GAT CTA AAA ATT GAA ATT TTA TTT TTT TTT TTT GGA ATA TAA ATA GAG CT-3′) (SEQ ID NO: 19) and oYF-40 (5′-CTA TTT ATA TTC CAA AAA AAA AAA ATA AAA TTT CAA TTT TTA GAT CCA-3′) (SEQ ID NO: 20). The sequence of the gene cassette is set out in SEQ ID NO: 3. Construction and purification of recombinant MVA-YF carrying the YFV prME gene cassette under the control of the selP promoter was performed as described in paragraph [0036]. Instead of the pd3-lacZ-mH5-YFprMEco plasmid, the pd3-lacZ-selP-YFprMEco plasmid was used for the transfection.

As another alternative, to direct the foreign gene into the HA locus of MVA, the YFprMEco cassette under the control of mH5 or selP promoter, respectively was inserted into the transfer plasmid pHA-vA (Scheiflinger et al. 1998) between the XhoI/SnaBI restriction sites resulting in the plasmids pHA-mH5-YFprMEco or pHA-selP-YFprMEco, respectively. Homologous recombination was performed in the same manner to generate recombinant MVA-YF with the alternate insertion site termed MVA-mHSYF or MVA-selPYF.

The absence of wild-type MVA from the recombinant virus was confirmed by PCR analysis and by a double immunostain assay. For PCR analysis, a primer pair was selected that binds in the flanking region to the deletion III integration sites resulting in a fragment of 3490 bp with MVA-YF, and in a fragment of 1298 bp with the wild-type MVA. This assay confirmed that the recombinant MVA-YF stock was free from parental wild-type virus at a detection limit of about 1 PFU contaminants among 1000 PFU of recombinant virus (data not shown).

To detect potential contaminating wild-type virus or recombinants that lost the ability to express YF antigen, DF-1 cells or cVero22 cells (Mayrhofer et al. 2009) were cultivated in 6 well plates and infected with 10, 100 or 1000 PFU of the recombinants. Wild-type virus and a mixture of wild-type virus and the respective recombinant were used as controls. After 1 h of incubation at 37° C. in 5% CO2, the viral inoculum was aspirated, and 3 ml of a 0.5% carboxymethylcellulose overlay with DMEM, supplemented with 5% FCS, was added to each well. After 4 days of incubation, the overlay was removed and the cells were fixed with methanol/aceton (1:1). To detect plaques of YFV E-protein expressors, a gp antiserum against YFV-17D was used. Goat anti-guinea pig horseradish peroxidase conjugated IgG (Jackson ImmunoResearch Laboratories, Inc.) was used as a secondary antibody. Plaques were visualized with diaminobenzidine (DAB) solutions including nickel (Vector Laboratories), resulting in black plaques. To detect MVA plaques without prME expression, a polyclonal rabbit anti-vaccinia virus serum was used (lot no. AVVSKP26012006). The secondary antibody was a goat anti-rabbit peroxidase conjugated IgG (Jackson Inc). Plaques were visualized with DAB solution without nickel, resulting in brown plaques. Black and brown plaques were counted visually.

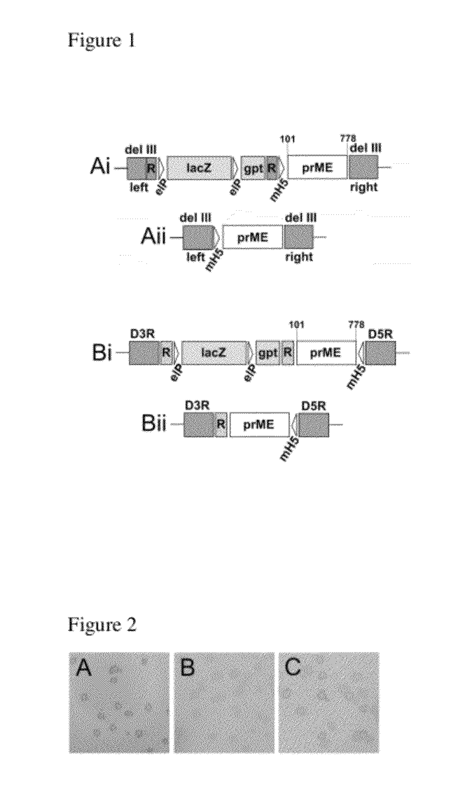

The MVA-YF infected cells showed uniformly black foci representing recombinants expressing prME proteins (FIG. 2A) indicating that the stock was free from wild-type MVA or any aberrant recombinants without prME expression (non-expressors). In the wild-type MVA control only brown foci were seen (FIG. 2B), whereas the MVA-YF/MVA spike control contained clearly distinguishable brown and black foci in the expected proportion (FIG. 2C).

### Example 2

### Construction and Characterization of the Recombinant Virus dVV-YF

In parallel, a D4R-defective vaccinia virus (dVV) expressing the codon-optimized prME CDS was generated, and was termed dVV-YF. For this purpose, the mH5-prME cassette was inserted into the plasmid pDW2 resulting in pDW-mH5-YFprMEco (FIG. 1 Bi).

For the generation of D4R-defective vaccinia viruses, a derivate of the plasmid pDW-2 (Holzer et al. 1998) was constructed. pDW-2 contains vaccinia virus genomic sequences of the D3R and D5R genes for homologous recombination and a lacZ/gpt marker cassette located between tandem DNA repeats allowing transient selection and blue plaque screening. The synthetic mH5-YFprMEco gene cassette was inserted into the XhoI/NotI site of plasmid pDW-2 resulting in pDW-mH5-YFprMEco (FIG. 1 Bi). The sequence of the promoter and prME gene cassette was verified by sequence analysis.

This plasmid was used to construct the non-replicating virus dVV-YF, in which the YFV prME expression cassette is inserted between the vaccinia D3R and D5R genes, replacing the essential D4R gene. Recombinant virus was generated by infecting D4R-complementing cVero22 cells with wild-type VV (strain Lister/Elstree) (VR-862 from the American Type Culture Collection), transfection of the recombination plasmid, and several rounds of plaque purification. dVV-YF (FIG. 1B ii). To generate the recombinant, replication-deficient vaccinia virus (dVV-YF) twenty micrograms of pDW2-mH5-YFprMEco were transfected into vaccinia virus Lister/Elstree infected cVero22 cells (Mayrhofer et al. 2009). Plaque purifications were done as described earlier (Holzer and Falkner 1997). A purified isolate of the defective dVV-YF obtained by this procedure was amplified to large scale in cVero cells and subjected to further characterization.

These steps resulted in a replication deficient recombinant virus, termed dVV-YF (FIG. 1B ii). The recombinant had the intended genetic structure without any marker gene, as characterized by PCR. It was growth incompetent in wild-type cells, and all plaques analyzed by double immunostaining expressed prME proteins (data not shown).

### Example 3

### Antigen Expression in Permissive Cells

The prME expression pattern by MVA-YF was first tested under conditions that are permissive for MVA replication. For this purpose, avian DF-1 cells were infected with a MVA-YF or with wild-type MVA or YFV-17D (17D) (commercially available vaccine Stamaril, Sanofi/Pasteur) as controls at a MOI of 0.01. Infected cells were incubated for four days and total cell lysates were investigated by SDS-PAGE and Western blot analysis using polyclonal anti-YFV-17D antiserum.

Expression of the prME protein by the MVA-YF and dVV-YF recombinants was assessed by Western blotting. To analyse the expression under permissive conditions, DF-1 cells or, in the case of the defective recombinant, cVero cells were infected with a MOI of 0.01 for 4 days. For the analysis under non-permissive conditions, HeLa or Sol8 cells were infected at a MOI of 10 for 72 h. Cells were infected in parallel with the corresponding wild-type vaccinia viruses or YFV-17D as controls. Sonicated and heat treated cell lysates were loaded on 12% polyacrylamide gels (Bio-Rad, Inc) and blotted onto nitrocellulose membrane (Invitrogen, Inc). To detect the prME protein, a polyclonal guinea pig (gp) antiserum against YFV-17D was used. Goat anti-guinea pig horseradish peroxidase conjugated IgG (Jackson ImmunoResearch Laboratories, Inc.) was used as a secondary antibody. YFV-17D infected HeLa cells (MOI 0.01 for 3 days) served as a positive control.

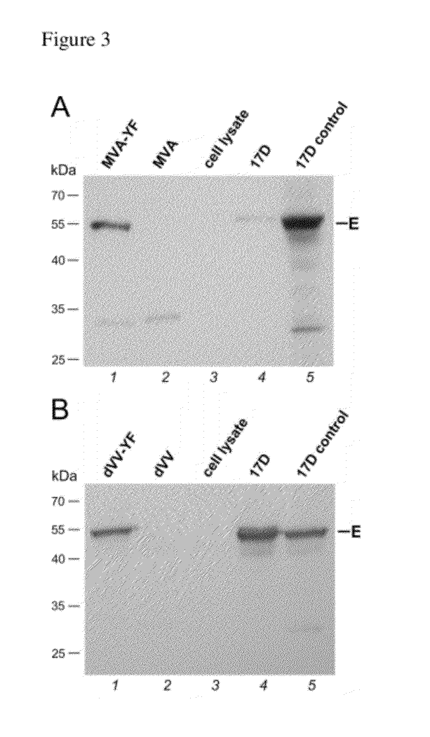

As shown in FIG. 3A, the YF envelope (E) protein expressed by the recombinant MVA-YF (lane 1) appeared as a single band in the 50 kDa size range, which is the expected size of flavivirus E proteins (Lindenbach B D, Thiel H J, and Rice C M 2007). An identical band was also detectable in the 17D control (lane 5). The E protein expression level of the recombinant MVA was higher than in the YFV-17D infection (lane 4). The low expression level of YFV-17D in avian DF-1 cells was seen repeatedly.

The expression patterns of the recombinant MVA-YF with the YFprMEco cassette in the HA locus were also studied in a human cell line. To analyze the prMEco expression under the control of the mH5 or selP promoter, respectively human (HeLa) cells were infected in duplicates with a MOI of 10 of MVA-selPYF. Infected cells were incubated for one day and total cell lysates were investigated by SDS-PAGE and Western blot analysis using anti-YFV antiserum. In HeLa cells, comparable amounts of E-protein were found in MVA-mHSYF and MVA-selPYF infections (data not shown).

To investigate the prME expression by dVV-YF in a cell culture system that supports replication of D4R-defective vaccinia, the complementing Vero cell line cVero22 was infected with a MOI of 0.01 with dVV-YF or with the dVV wild-type virus or YFV-17D as controls. Further steps were performed as described above.

As shown in FIG. 3B the recombinant dVV-YF (lane 1) expressed the E protein in the infected cVero22 cells, as did the YFV-17D virus (lane 4).

### Example 4

### Antigen Expression in Non-Permissive Cells

The recombinant MVA-YF and dVV-YF were designed for human use for inducing an immune response, and efficacy was assessed in a mouse protection model. In the mouse and human organisms, these viruses do not replicate. Despite of the absence of viral replication, YFV protein expression should take place at reasonable levels for the induction of an efficient immune response. For this reason, the expression patterns were also studied in a human and in a mouse cell line, non-permissive for both the recombinant MVA-YF and dVV-YF. Mouse muscle (Sol8) or human (HeLa) cells were infected with a MOI of 10 of MVA-YF or dVV-YF and with the corresponding controls. Infected cells were incubated for two days and total cell lysates were investigated by SDS-PAGE and Western blot analysis using anti-YFV antiserum.

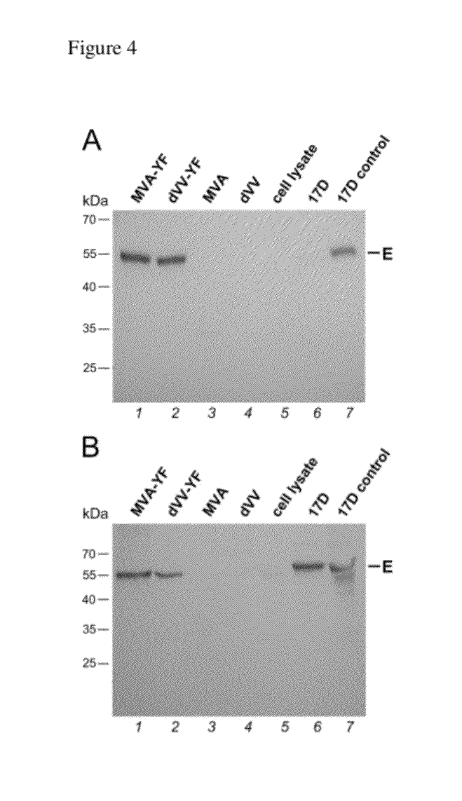

The expression in Sol8 muscle cells should reflect the target cell type in the selected mouse challenge model in which mice are immunized intramuscularly (i.m.). As shown in FIG. 4A, recombinant MVA-YF (lane 1) and dVV-YF (lane 2) expressed the E-protein in comparable amounts. As expected in the negative controls (lanes 3-5) no YFV protein was detectable. In this setting, E-protein expression through the YFV-17D positive control (lane 6) was below the limit of detection.

In HeLa cells (FIG. 4B) again comparable amounts of E-protein were found in MVA-YF (lane 1) and dVV-YF (lane 2) infections. The YFV-17D infected cells (lane 6) revealed comparable amounts of E-protein. Thus, in humans vaccinated with non-replicating MVA-YF or dVV-YF, correct expression of the YFV antigen at significant levels can be expected.

### Example 5

### Protection Studies in Mice

Next, the capacity of recombinant MVA-YF and dVV-YF to protect mice against a lethal i.c. challenge with YFV-17D virus was analyzed. All animal experiments were reviewed by the Institutional Animal Care and Use Committee (IACUC) and approved by the Austrian regulatory authorities. All animal experiments were conducted in accordance with Austrian laws on animal experimentation and guidelines set out by the Association for Assessment and Accreditation of Laboratory Animal Care (AAALAC). Animals were housed in facilities accredited by the AAALAC.

Groups of six Balb/c mice (Charles River) were immunized with a single intramuscular injection of MVA-YF or dVV-YF over a dose range of 1×10² to 1×10⁵ TCID₅₀ in a volume of 50 μl in PBS-0.01% human serum albumin (HSA) buffer. Control groups were immunized with 1×10⁶ TCID₅₀ of wild-type MVA, dVV or PBS as negative controls, and with 1×10⁴ TCID₅₀ (equivalent to the human dose) of YFV-17D as positive control in a volume of 50 μl in PBS-0.01% HSA or with PBS buffer. Mice were challenged at day 21 post vaccination intracerebrally (i.c.) with 1×10⁵ TCID₅₀ (>1000 mouse lethal dose 50 (LD₅₀)) of YFV-17D in TBS-0.01% HSA buffer and monitored for either 14 or 21 days for clinical symptoms and survival. The LD₅₀ in nine week old mice was determined to be approximately 83 TCID₅₀ (data not shown).

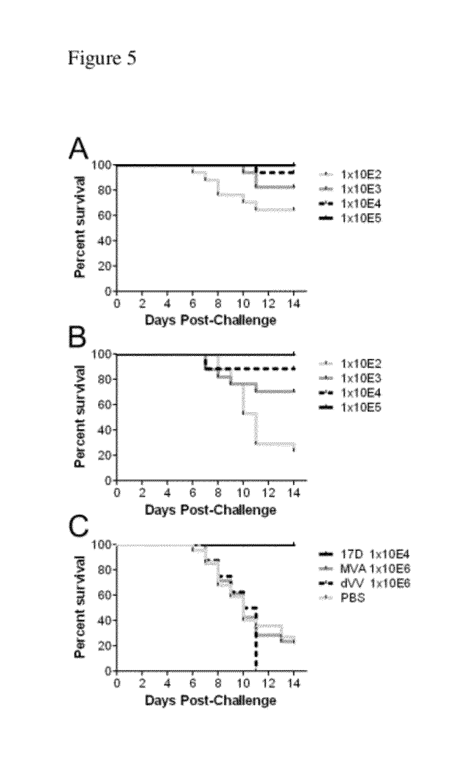

Protection by MVA-YF was clearly dose-dependent (FIG. 5A). The highest dose of 1×10⁵ TCID₅₀ per animal conferred full protection, and even the lowest dose of 1×10² protected more than 50% of the animals. Also, in the dVV-YF groups (FIG. 5B) protection was dose-dependent with 100% survival at the highest immunization dose of 1×10⁵ TCID₅₀. However, the lower doses did not protect as well as the recombinant MVA-YF. A dose of 1×10² TCID₅₀ protected less than 30%. All negative control groups, injected with the wild-type vaccinia viruses or PBS, died or showed low survival rates of maximal 20%. As expected, complete protection was seen in groups immunized with YFV-17D (FIG. 5C).

In order to define the correlate of protection, neutralization antibody titers were analyzed on day 19 in pre-challenge sera. After vaccination with a single dose, the PRNT₅₀ titer was low. This was true for vaccinations with recombinant viruses up to the highest immunization dose, but also with YFV-17D. Therefore, a second experiment was performed in which mice received single or double dose inoculations of 10⁴, 10⁶, 10⁷ TCID₅₀ of MVA-YF or dVV-YF or 10⁴ and 10⁶ TCID₅₀ of YFV-17D virus as a positive control. Additionally, mice were immunized with a double dose of 1×10⁷ TCID₅₀ of the empty MVA or dVV vectors as negative controls.

Sera were collected at day 42 after the primary immunization and analyzed for YFV neutralizing antibodies by PRNT₅₀ assay. Approximately 3×10⁵ Vero cells were seeded per well in 6 well plates and cultured overnight to obtain confluent monolayers. Sera were complement-inactivated at 56° C. for 30 min. Pre-vaccination sera was tested in 1:10 dilution, to which 100 PFU of YFV-17D were added. Serial two-fold dilutions of the post-vaccination sera were mixed with 100 PFU of YFV-17D strain and incubated overnight at 4° C. The mixture of virus and serum were added to the Vero cell monolayers and incubated for 1 hour at 37° C. Virus/serum mixtures were replaced by 0.75% carboxymethylcellulose-DMEM solution, incubated for 4 days and visualized with immunostaining as described above. The neutralizing antibody titer is the reciprocal of the highest serum dilution that reduced the number of viral plaques by at least 50% relative to the pre-vaccination sera.

As shown in Table 1 below both recombinant vaccines induced 100% protection after one application of 10⁵ TCID₅₀, however no or only a low PRNT₅₀ titers were measureable even at the highest dose of 10⁷ TCID₅₀. Only the YFV-17D vaccine induced measurable neutralization titers after a single dose administration of 10⁴ and 10⁶ TCID₅₀.

TABLE 1
Protection and pre-challenge YFV PRNT in mice

| | | Protection | | | | | |
|---|---|---|---|---|---|---|---|
| | Immun | [survivors/ | | PRNT₅₀ | | PRNT₅₀ | |
| | Dose | total (%)]³ | | [GMT]^{1,2} | | [GMT]^{1,2} | |
| Vaccine | [log10] | Single dose | | Single dose | | Double dose | |
| MVA- | 2 | 11/17 | (64.7) | n.d.⁴ | | n.d.⁴ | |
| YF | 3 | 14/17 | (82.4) | n.d.⁴ | | n.d.⁴ | |
| | 4 | 16/17 | (94.1) | <10 | (<10)⁵ | 17 | (10-20)⁵ |
| | 5 | 17/17 | (100) | n.d. | | n.d.⁴ | |
| | 6 | n.d.⁴ | | <10 | (<10-20)⁵ | 42 | (20-80)⁵ |
| | 7 | n.d.⁴ | | <10 | (<10-10)⁵ | 80 | (40-160)⁵ |
| dVV- | 2 | 4/17 | (23.5) | n.d.⁴ | | n.d.⁴ | |
| YF | 3 | 12/17 | (70.6) | n.d.⁴ | | n.d.⁴ | |
| | 4 | 16/18 | (88.9) | <10 | (<10)⁵ | 18.2 | (10-40)⁵ |
| | 5 | 18/18 | (100) | n.d.⁴ | | n.d.⁴ | |
| | 6 | n.d.⁴ | | 17.3 | (10-20)⁵ | 26.2 | (10-40)⁵ |
| | 7 | n.d.⁴ | | <10 | (<10-10)⁵ | 33 | (15-160)⁵ |
| 17D | 4 | 17/17 | (100) | 13.2 | (<10-40)⁵ | 120 | (80-160)⁵ |

| | | | | | |
|---|---|---|---|---|---|
| 6 | — | 54.3 | (20-80) | 381 | (160-640)⁵ |

| | | | | | |
|---|---|---|---|---|---|
| MVA | 6 | 5/21 | (23.8) | <10 | <10 |
| dVV | 6 | 8/8 | (0) | <10 | <10 |
| Buffer | — | 8/22 | (22.7) | <10 | <10 |

¹Geometric mean titer
²Results of two independent experiments
³Results of three independent experiments (except dVV)
⁴Not determined
⁵Range of PRNT50

After a second vaccination with MVA YF and dVV YF neutralization titers were detectable in a dose dependent fashion. Here, the MVA based vaccine showed in average somewhat higher titers than the dVV-YF vaccine. The highest neutralization titers were induced with the YFV-17D vaccine and no PRNT₅₀ was measurable in wild-type MVA and dVV immunized mice.

### Example 6

### Induction of Envelope Protein-Specific T Cell Responses in Mice

While induction of a humoral immune response and generation of neutralizing antibodies against the envelope protein represent the major protective mechanism following vaccination with the live YFV-17D vaccine (Monath 1986; Monath and Barrett 2003), the cellular immune responses are also thought to play an important role in protection against infection (Liu and Chambers 2001; Co et al. 2002; van der Most et al. 2002; Monath and Barrett 2003; Maciel, Jr. et al. 2008). Recently, the T cell responses induced by the YFV-17D vaccine were characterized (Maciel, Jr. et al. 2008). In this report, BALB/c (H2d) mice were inoculated with 17D vaccine strain and CD8- and CD4-specific epitopes were investigated (Maciel, Jr. et al. 2008).

To compare the T-cell responses following MVA-YF or dVV-YF vaccination to the YFV-17D vaccine, mice were immunized twice (0 and 3 weeks) with the vaccinia virus recombinants or the corresponding controls. Splenocytes were prepared on day 28 and stimulated in vitro with CD8- and CD4-specific peptides derived from YF envelope (Maciel, Jr. et al. 2008). The percentages of IFN-γ producing T cells were determined by a FACS-based intracellular cytokine assay. Mice were immunized as described above, spleens were collected at day 28 post-immunization, and splenocyte cell suspensions were prepared. Vaccine-specific cell-mediated immunity was evaluated as described previously (Mayrhofer et al. 2009) using flow cytometric IFN-γ response assays and analysis of killing of peptide-pulsed target cells by specific CD8 T cells. Splenocytes were restimulated using the following previously described (Maciel, Jr. et al. 2008) synthetic peptides from the yellow fever envelope protein: E57-71, E129-143, E133-147 (15 mer peptides recognized by CD4 T cells) and E60-68, E330-338, E332-340 (9 mer peptides recognized by CD8 T cells).

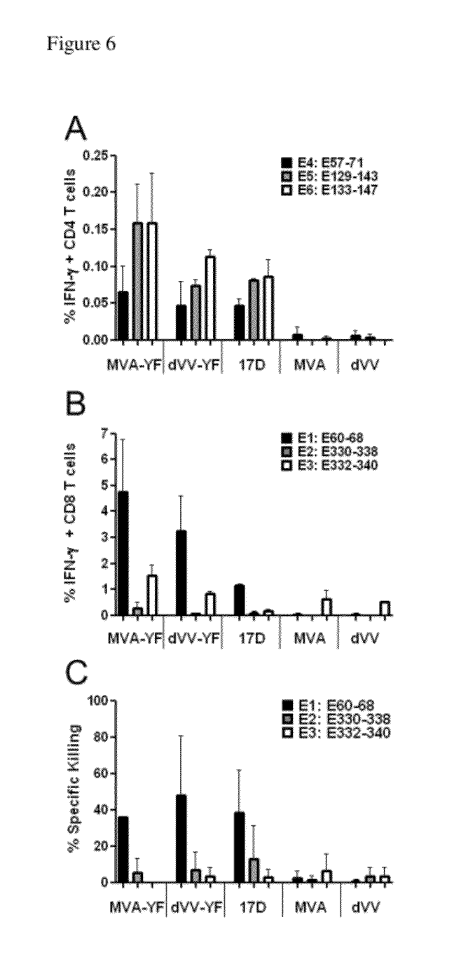

The results of CD4-specific response (Th1) of two independent experiments obtained with the prME expressing recombinants MVA-YF, dVV-YF and with the corresponding controls are shown in FIG. 6A. Following stimulation with peptides E4-E6, recombinant MVA-YF induced the highest frequency of CD4 positive IFN-γ producing T cells, whereas dVV-YF and YFV-17D induces somewhat lower but generally comparable amounts of specific CD4 T cells. As expected, the wild-type controls did not induce a significant response.

The frequency of vaccine-specific CD8 T cells induced by the recombinants and controls upon in vitro stimulation with the E peptides are shown in FIG. 6B. Up to 5% of the CD8 T cells responded to the immunodominant peptide E1. The highest frequency of vaccine-specific CD8 T cells were detected in the mice immunized with the MVA recombinant, followed by dVV-YF. CD8 T cell activation by the YFV-17D vaccine was at a level much lower than by the recombinants.

To verify that the envelope specific CD8 T cells were functionally active and kill target cells pulsed with specific YFV envelope peptides, a cytotoxic T-lymphocyte (CTL) killing assay based on fluorometric techniques was used (Hermans et al. 2004). For this purpose, splenocytes were incubated with peptide-presenting and dye-labeled target and control cells. The reduction of the peptide-pulsed target cells versus control cells after incubation with the splenocytes indicates the presence of functional CTLs. Significant CTL-specific killing (FIG. 6C) was induced only in E1 pulsed target cells. Killing was comparable for the groups immunized with MVA-YF (36%±0), dVV-YF (48%±23) and YFV-17D (38.5%±16.5). In summary, immunization with the MVA and dVV recombinants and with YFV-17D vaccine induced functionally competent CTLs.

### Example 7

### Influence of Pre-Existing Immunity on Protection

Assuming that a subset of the human population possess immunity to vaccinia virus due to previous vaccinations, either those having received smallpox vaccination or being vaccinated with MVA recombinant vaccines (Cebere et al. 2006; Bejon et al. 2007; Harrop et al. 2008; Brookes et al. 2008), it is important to analyze the influence of an existing immunity to the vector on the protection by the recombinant vaccine. To investigate if previous exposure to vaccinia virus influences the effectiveness of the recombinants, Balb/c mice were immunized first with 2×10⁶ TCID₅₀ wild-type MVA (single and double dose) or vaccinia virus Lister/Elstree, respectively. Three months later, animals were vaccinated with a suboptimal single or double dose of 1×10³ TCID₅₀ or with a usually protective dose of 1×10⁵ TCID₅₀ of MVA-YF, dVV-YF, and the corresponding controls. Animals were finally challenged with more than a 1.000-fold LD₅₀ YFV-17D. The design of the experiment and the results are outlined in Table 2 below. Before immunzation with MVA-YF, sera were collected to determine vaccinia virus-specific neutralizing antibody titers (PRNT₅₀). The test for neutralizing antibodies against vaccinia virus was performed as described above, with the difference that vaccinia virus strain Lister/Elstree (ATCC VR 862) was used as the target virus, and neutralization was done at 37° C. for 1 hour. VV plaques were stained with crystal violet.

TABLE 2
Immunization schema, VV PRNT prior to YF vaccination, survival in mice

| | | 1st Pre- | 2nd Pre- | | | VV PRNT₅₀ | No. of |
|---|---|---|---|---|---|---|---|
| | | Immun | Immun. | 1st Immun. | 2nd Immun. | GMT¹ | survivors/ |
| | | [day 0] | [day 21] | [day 84] | [day 104] | [day 82] | total (%) |
| | 1³ | VV-Lister | — | MVA-YF (10³) | — | 20 | 5/6 (83.3) |
| A | 2² | MVA | — | MVA-YF (10³) | — | 1280¹ | 5/10 (50) |
| | 3³ | MVA | MVA | MVA-YF (10³) | — | 640 | 1/6 (16.7) |
| | 4² | — | — | MVA-YF (10³) | — | <20¹ | 0/12 (0) |
| | 5³ | VV-Lister | — | MVA-YF (10³) | MVA-YF (10³) | 160 | 3/6 (50) |
| B | 6² | MVA | | MVA-YF (10³) | MVA-YF (10³) | 640¹ | 7/12 (58.3) |
| | 7³ | MVA | MVA | MVA-YF (10³) | MVA-YF (10³) | 640 | 3/5 (60) |
| | 8² | — | — | MVA-YF (10³) | MVA-YF (10³) | <20¹ | 7/12 (58.3) |
| C | 9³ | MVA | MVA | MVA-YF (10⁵) | — | 320 | 5/5 (100) |
| | 10³ | — | — | MVA-YF (10⁵) | — | <20 | 5/5 (100) |
| D | 11² | — | — | 17D (10⁴) | — | <20 | 10/12 (83.3) |
| | 12² | — | — | Buffer | — | <20 | 1/10 (10) |

¹Geometric mean titer
²Results of two independent experiments
³Results of one experiment

All animals which obtained a single suboptimal dose of MVA-YF without pre-vaccination (FIG. 7A), died after challenge (Tab. 2, group 4). Interestingly, mice pre-vaccinated with wild-type vaccinia viruses (groups 1-3) showed increased protection compared to animals without pre-immunization (group 4). The best protection was induced in animals vaccinated with vaccinia virus Lister/Elstree strain (83%; group 1), followed by groups obtained single (50%; group 2) or double (17%; group 3) dose MVA wild-type. However, no correlation was observed between the vaccinia virus-specific neutralizing antibody titers (Table 2) and the degree of protection.

In the groups immunized with a double dose of MVA-YF, no effect of the different pre-vaccinations with wild-type viruses was seen (FIG. 7B, Tab. 2, groups 5-8). Comparable protection was achieved in the groups without (58% survival) or with pre-existing immunity (VV PRNT₅₀ 160-800, 50-60% survival). It was further confirmed, that the optimal dose of 1×10⁵ TCID₅₀ MVA-YF was still able to induce 100% protection despite a pre-existing immunity (VV PRNT₅₀ 320) against MVA (groups 9, 10). Therefore, a pre-existing anti-vaccinia virus immunity does not have a negative influence on the protection of MVA-YF against a lethal YFV-17D challenge.

### Example 8

### Safety of MVA-YF and dVV-YF

Considering the possibility that the introduction of the prME gene might alter the infectivity of the vaccinia virus vectors, the safety profile of the vaccines was tested. For this purpose, Balb/c mice were challenged i.c. with high doses of 1×10⁵ to 1×10⁷ TCID₅₀ MVA-YF, dVV-YF and with the corresponding wild-type viruses. To compare also the safety profile of the recombinants with the YFV-17D vaccine, 1×10¹ to 1×10³ TCID₅₀ of YFV-17D were administered intracerebrally.

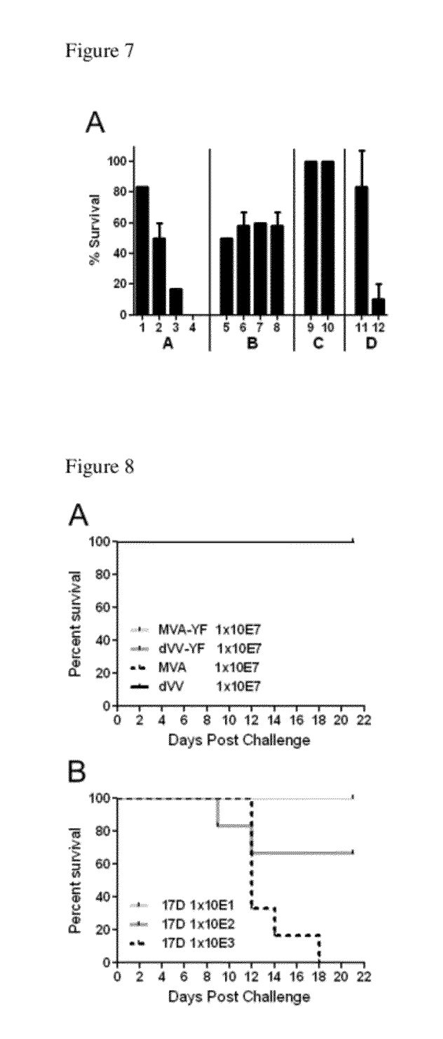

In the vaccinia virus challenged groups, complete survival was seen even with the highest dose of 1×10⁷ TCID₅₀ (FIG. 8 A). Furthermore, there was no difference between the wild-type vaccinia vectors and the recombinants. In contrast to YFV-17D challenged mice, a very low dose of 1×10² TCID₅₀ YFV-17D induced 65% lethality and 1×10³ TCID₅₀ killed 100% of the mice (FIG. 8 B). In conclusion the non-replicating vaccinia-based vaccines are safe and very high doses do not kill mice. Furthermore the introduction of the prME gene did not altered the safety profile of the vaccinia vectors while low doses of the YFV-17D vaccine killed the mice after i.c. administration.

The present invention is illustrated by the foregoing examples and variations thereof will be apparent to those skilled in the art. Therefore, no limitations other than those set out in the following claims should be placed on the invention.

### LITERATURE CITED

A. Mayr, V. Hochstein-Mintzel, and H. Stickl. Abstammung, Eigenschaften and Verwendung des attenuierten Vaccinia-Stammes MVA. Infection 3 (1975) Nr.1.
Akondy, R. S., N. D. Monson, J. D. Miller, S. Edupuganti, D. Teuwen, H. Wu, F. Quyyumi et al. 2009. The yellow fever virus vaccine induces a broad and polyfunctional human memory CD8+ T cell response. J Immunol 183, no. 12:7919-7930.
Antoine, G., F. Scheiflinger, F. Dorner, and F. G. Falkner. 1998. The complete genomic sequence of the modified vaccinia Ankara strain: comparison with other orthopoxviruses. Virology 244, no. 2:365-396.
Antoine, G., F. Scheiflinger, G. Holzer, T. Langmann, F. G. Falkner, and F. Dorner. 1996. Characterization of the vaccinia MVA hemagglutinin gene locus and its evaluation as an insertion site for foreign genes. Gene 177, no. 1-2:43-46.
Barrett, A. D. 1997. Yellow fever vaccines. Biologicals 25, no. 1:17-25.
Bejon, P., E. Ogada, T. Mwangi, P. Milligan, T. Lang, G. Fegan, S. C. Gilbert et al. 2007. Extended follow-up following a phase 2b randomized trial of the candidate malaria vaccines FP9 ME-TRAP and MVA ME-TRAP among children in Kenya. PLoS.One. 2, no. 1:e707.
Belsher, J. L., P. Gay, M. Brinton, J. DellaValla, R. Ridenour, R. Lanciotti, A. Perelygin et al. 2007. Fatal multiorgan failure due to yellow fever vaccine-associated viscerotropic disease. Vaccine 25, no. 50:8480-8485.
Brandriss, M. W., J. J. Schlesinger, and E. E. Walsh. 1990. Immunogenicity of a purified fragment of 17D yellow fever envelope protein. J. Infect. Dis. 161, no. 6:1134-1139.
Brandriss, M. W., J. J. Schlesinger, E. E. Walsh, and M. Briselli. 1986. Lethal 17D yellow fever encephalitis in mice. I. Passive protection by monoclonal antibodies to the envelope proteins of 17D yellow fever and dengue 2 viruses. J. Gen. Virol. 67 (Pt 2):229-234.
Brookes, R. H., P. C. Hill, P. K. Owiafe, H. B. Ibanga, D. J. Jeffries, S. A. Donkor, H. A. Fletcher et al. 2008. Safety and immunogenicity of the candidate tuberculosis vaccine MVA85A in West Africa. PLoS One. 3, no. 8:e2921.
Carroll, M. W. and B. Moss. 1997. Host range and cytopathogenicity of the highly attenuated MVA strain of vaccinia virus: propagation and generation of recombinant viruses in a nonhuman mammalian cell line. Virology 238, no. 2:198-211.
Cebere, I., L. Dorrell, H. McShane, A. Simmons, S. McCormack, C. Schmidt, C. Smith et al. 2006. Phase I clinical trial safety of DNA- and modified virus Ankara-vectored human immunodeficiency virus type 1 (HIV-1) vaccines administered alone and in a prime-boost regime to healthy HIV-1-uninfected volunteers. Vaccine 24, no. 4:417-425.
Chakrabarti, S., J. R. Sisler, and B. Moss. 1997. Compact, synthetic, vaccinia virus early/late promoter for protein expression. Biotechniques 23, no. 6:1094-1097.
Chan, R. C., D. J. Penney, D. Little, I. W. Carter, J. A. Roberts, and W. D. Rawlinson. 2001. Hepatitis and death following vaccination with 17D-204 yellow fever vaccine. Lancet 358, no. 9276:121-122.
Co, M. D., M. Terajima, J. Cruz, F. A. Ennis, and A. L. Rothman. 2002. Human cytotoxic T lymphocyte responses to live attenuated 17D yellow fever vaccine: identification of HLA-B35-restricted CTL epitopes on nonstructural proteins NS1, NS2b, NS3, and the structural protein E. Virology 293, no. 1:151-163.
Doblas, A., C. Domingo, H. G. Bae, C. L. Bohorquez, Ory F. de, M. Niedrig, D. Mora, F. J. Carrasco, and A. Tenorio. 2006. Yellow fever vaccine-associated viscerotropic disease and death in Spain. J Clin Virol. 36, no. 2:156-158.
Drexler, I., K. Heller, B. Wahren, V. Erfle, and G. Sutter. 1998. Highly attenuated modified vaccinia virus Ankara replicates in baby hamster kidney cells, a potential host for virus propagation, but not in various human transformed and primary cells. J. Gen. Virol. 79 (Pt 2):347-352.
Drexler, I., C. Staib, and G. Sutter. 2004. Modified vaccinia virus Ankara as antigen delivery system: how can we best use its potential? Curr. Opin. Biotechnol. 15, no. 6:506-512.
Gerasimon, G. and K. Lowry. 2005. Rare case of fatal yellow fever vaccine-associated viscerotropic disease. South. Med. J 98, no. 6:653-656.
Gubler DJ, Kuno G, and Markoff L. Flaviviruses. In Knipe D M, Howley P M, editors. Fields Virology. 5th ed. Philadelphia, Pa.: Lippincott Williams & Wilkins 2007. 2007.

### Ref Type: Generic

Harrop, R., N. Drury, W. Shingler, P. Chikoti, I. Redchenko, M. W. Carroll, S. M. Kingsman et al. 2008. Vaccination of colorectal cancer patients with Tro Vax given alongside chemotherapy (5-fluorouracil, leukovorin and irinotecan) is safe and induces potent immune responses. Cancer Immunol Immunother. 57, no. 7:977-986.
Hermans, I. F., J. D. Silk, J. Yang, M. J. Palmowski, U. Gileadi, C. McCarthy, M. Salio, F. Ronchese, and V. Cerundolo. 2004. The VITAL assay: a versatile fluorometric technique for assessing CTL- and NKT-mediated cytotoxicity against multiple targets in vitro and in vivo. J Immunol Methods 285, no. 1:25-40.
Holzer, G. W. and F. G. Falkner. 1997. Construction of a vaccinia virus deficient in the essential DNA repair enzyme uracil DNA glycosylase by a complementing cell line. J. Virol. 71, no. 7:4997-5002.
Holzer, G. W., W. Gritschenberger, J. A. Mayrhofer, V. Wieser, F. Dorner, and F. G. Falkner. 1998. Dominant host range selection of vaccinia recombinants by rescue of an essential gene. Virology 249, no. 1:160-166.
Holzer, G. W., G. Remp, G. Antoine, M. Pfleiderer, O. M. Enzersberger, W. Emsenhuber, T. Hammerle, F. Gruber, C. Urban, F. G. Falkner and F. Dorner. 1999. Highly efficient induction of productive immunity by a vaccinia virus vector defective in late gene expression. J. Virol. 73, no. 6:4536-4542.
Kaufman, H. L., B. Taback, W. Sherman, D. W. Kim, W. H. Shingler, D. Moroziewicz, G. Deraffele et al. 2009. Phase II trial of Modified Vaccinia Ankara (MVA) virus expressing 5T4 and high dose Interleukin-2 (IL-2) in patients with metastatic renal cell carcinoma. J. Transl. Med. 7:2.
Kengsakul, K., K. Sathirapongsasuti, and S. Punyagupta. 2002. Fatal myeloencephalitis following yellow fever vaccination in a case with HIV infection. J. Med. Assoc. Thai. 85, no. 1:131-134.
Lindenbach B D, Thiel H J, and Rice C M. Flaviviridae: The viruses and their replication. In Knipe D M, Howley P M, editors. Fields Virology. 5th ed. Philadelphia, Pa.: Lippincott Williams & Wilkins. 2007.
Lindsey, N. P., B. A. Schroeder, E. R. Miller, M. M. Braun, A. F. Hinckley, N. Marano, B. A. Slade et al. 2008. Adverse event reports following yellow fever vaccination. Vaccine 26, no. 48:6077-6082.
Liu, T. and T. J. Chambers. 2001. Yellow fever virus encephalitis: properties of the brain-associated T-cell response during virus clearance in normal and gamma interferon-deficient mice and requirement for CD4+ lymphocytes. J. Virol. 75, no. 5:2107-2118.
Maciel, M., Jr., S. N. Kellathur, P. Chikhlikar, R. Dhalia, J. Sidney, A. Sette, T. J. August, and E. T. Marques, Jr. 2008. Comprehensive analysis of T cell epitope discovery strategies using 17DD yellow fever virus structural proteins and BALB/c (H2d) mice model. Virology 378, no. 1:105-117.
Mackett, M., G. L. Smith, and B. Moss. 1982. Vaccinia virus: a selectable eukaryotic cloning and expression vector. Proc. Natl. Acad. Sci. U.S.A 79, no. 23:7415-7419.
Martin, M., T. F. Tsai, B. Cropp, G. J. Chang, D. A. Holmes, J. Tseng, W. Shieh et al. 2001. Fever and multisystem organ failure associated with 17D-204 yellow fever vaccination: a report of four cases. Lancet 358, no. 9276:98-104.
Mayr, A., H. Stickl, H. K. Muller, K. Danner, and H. Singer. 1978. [The smallpox vaccination strain MVA: marker, genetic structure, experience gained with the parenteral vaccination and behavior in organisms with a debilitated defence mechanism (author's transl)]. Zentralbl. Bakteriol. B 167, no. 5-6:375-390.
Mayrhofer, J., S. Coulibaly, A. Hessel, G. W. Holzer, M. Schwendinger, P. Bruhl, M. Gerencer et al. 2009. Nonreplicating vaccinia virus vectors expressing the H5 influenza virus hemagglutinin produced in modified Vero cells induce robust protection. J. Virol. 83, no. 10:5192-5203.
Meyer, H., G. Sutter, and A. Mayr. 1991. Mapping of deletions in the genome of the highly attenuated vaccinia virus MVA and their influence on virulence. J. Gen. Virol. 72 (Pt 5):1031-1038.
Miller, J. D., R. G. van der Most, R. S. Akondy, J. T. Glidewell, S. Albott, D. Masopust, K. Murali-Krishna et al. 2008. Human effector and memory CD8+ T cell responses to smallpox and yellow fever vaccines. Immunity. 28, no. 5:710-722.
Monath T. P. Yellow Fever. “Vaccines” (S. A. Plotkin, and W. A. Orenstein, Eds.) 4th ed., 1095-1176. 2004.

### Ref Type: Generic

Monath, T. P. The Togaviridae and Flaviviridae. In: Schleisinger S., Schleisinger M. J. (EDS.), Pathology of the Flavivirus Plenum Press, New York, 375-440. 1986.

### Ref Type: Generic

2007. Dengue and yellow fever—challenges for the development and use of vaccines. N Engl. J. Med. 357, no. 22:2222-2225.
2001. Yellow fever: an update. Lancet Infect. Dis. 1, no. 1:11-20.
Monath, T. P. and A. D. Barrett. 2003. Pathogenesis and pathophysiology of yellow fever. Adv. Virus Res. 60:343-395.
Monath, T. P., C. K. Lee, J. G. Julander, A. Brown, D. W. Beasley, D. M. Watts, E. Hayman et al. 2010. Inactivated yellow fever 17D vaccine: development and nonclinical safety, immunogenicity and protective activity. Vaccine 28, no. 22:3827-3840.
Nam, J. H., L. S. Wyatt, S. L. Chae, H. W. Cho, Y. K. Park, and B. Moss. 1999. Protection against lethal Japanese encephalitis virus infection of mice by immunization with the highly attenuated MVA strain of vaccinia virus expressing JEV prM and E genes. Vaccine 17, no. 3:261-268.
Ober, B. T., P. Bruhl, M. Schmidt, V. Wieser, W. Gritschenberger, S. Coulibaly, H. Savidis-Dacho, M. Gerencer, and F. G. Falkner. 2002. Immunogenicity and safety of defective vaccinia virus lister: comparison with modified vaccinia virus Ankara. J. Virol. 76, no. 15:7713-7723.
Pincus, S., P. W. Mason, E. Konishi, B. A. Fonseca, R. E. Shope, C. M. Rice, and E. Paoletti. 1992. Recombinant vaccinia virus producing the prM and E proteins of yellow fever virus protects mice from lethal yellow fever encephalitis. Virology 187, no. 1:290-297.
Rice, C. M., E. M. Lenches, S. R. Eddy, S. J. Shin, R. L. Sheets, and J. H. Strauss. 1985. Nucleotide sequence of yellow fever virus: implications for flavivirus gene expression and evolution. Science 229, no. 4715:726-733.
Scheiflinger, F., F. Dorner, and F. G. Falkner. 1998. Transient marker stabilisation: a general procedure to construct marker-free recombinant vaccinia virus. Arch. Virol. 143, no. 3:467-474.
Slavik, I., F. Ciampor, and V. Mayer. 1983. Optimalized conditions of tick-borne encephalitis virus production in vitro. Acta Virol. 27, no. 2:97-104.
Staib, C., I. Drexler, M. Ohlmann, S. Wintersperger, V. Erfle, and G. Sutter. 2000. Transient host range selection for genetic engineering of modified vaccinia virus Ankara. Biotechniques 28, no. 6:1137-6, 1148.
Stickl, H., V. Hochstein-Mintzel, A. Mayr, H. C. Huber, H. Schafer, and A. Holzner. 1974. [MVA vaccination against smallpox: clinical tests with an attenuated live vaccinia virus strain (MVA) (author's transl)]. Dtsch. Med. Wochenschr. 99, no. 47:2386-2392.
Stokes, A., J. H. Bauer, and N. P. Hudson. 2001. The transmission of yellow fever to Macacus rhesus. 1928. Rev Med. Virol. 11, no. 3:141-148.
Theiler, M. and H. H. Smith. 1937. The use of yellow fever virus modified by in vitro cultivation for human immunization. J. Exp. Med. 65, no. 6:787-800.
Tykodi, S. S. and J. A. Thompson. 2008. Development of modified vaccinia Ankara-5T4 as specific immunotherapy for advanced human cancer. Expert. Opin. Biol. Ther 8, no. 12:1947-1953.
van der Most, R. G., L. E. Harrington, V. Giuggio, P. L. Mahar, and R. Ahmed. 2002. Yellow fever virus 17D envelope and NS3 proteins are major targets of the antiviral T cell response in mice. Virology 296, no. 1:117-124.
Vasconcelos, P. F., E. J. Luna, R. Galler, L. J. Silva, T. L. Coimbra, V. L. Barros, T. P. Monath et al. 2001. Serious adverse events associated with yellow fever 17DD vaccine in Brazil: a report of two cases. Lancet 358, no. 9276:91-97.
WHO. Yellow fever. Yellow Fever fact sheet No 100 (http://www.who.int/mediacentre/factsheets/fs100/en/). 2009.
Wyatt, L. S., M. W. Carroll, C. P. Czerny, M. Merchlinsky, J. R. Sisler, and B. Moss. 1998. Marker rescue of the host range restriction defects of modified vaccinia virus Ankara. Virology 251, no. 2:334-342.
Wyatt, L. S., S. T. Shors, B. R. Murphy, and B. Moss. 1996. Development of a replication-deficient recombinant vaccinia virus vaccine effective against parainfluenza virus 3 infection in an animal model. Vaccine 14, no. 15:1451-1458.

1. A recombinant, modified vaccinia virus Ankara (MVA) comprising a gene cassette encoding a yellow fever virus (YFV) prME polypeptide.

2. The recombinant MVA of claim 1 wherein the gene cassette encodes a YFV prME amino acid sequence set out in SEQ ID NO: 2, SEQ ID NO: 5, SEQ ID NO: 6, SEQ ID NO: 7, SEQ ID NO: 8, SEQ ID NO: 9, or SEQ ID NO: 10.

3. The recombinant MVA of claim 1 wherein expression of the YFV prME polypeptide from the gene cassette is under the control of an mH5 promoter or selP promoter.

4. (canceled)

5. The recombinant MVA of claim 1 comprising the YFV-17D prME gene cassette set out in SEQ ID NO: 1.

6. The recombinant MVA of claim 1 comprising the YFV-17D prME gene cassette set out in SEQ ID NO: 3.

7. The recombinant MVA of claim 1 wherein the prME gene cassette is inserted in the MVA in the deletion I region, the deletion II region, the deletion III region, the deletion IV region, the thymidine kinase locus, the D4/5 intergenic region, or the HA locus.

8-12. (canceled)

13. A pharmaceutical composition comprising a recombinant, modified vaccinia virus Ankara (MVA) comprising a gene cassette encoding a yellow fever virus (YFV) prME polypeptide.

14. The pharmaceutical composition of claim 13 wherein the gene cassette encodes a YFV prME amino acid sequence set out in SEQ ID NO: 2, SEQ ID NO: 5, SEQ ID NO: 6, SEQ ID NO: 7, SEQ ID NO: 8, SEQ ID NO: 9, or SEQ ID NO: 10.

15. The pharmaceutical composition of claim 13 wherein expression of the YFV prME polypeptide from the gene cassette is under the control of an mH5 promoter or selP promoter.

16. (canceled)

17. The pharmaceutical composition of claim 13 comprising the YFV-17D prME gene cassette set out in SEQ ID NO: 1.

18. The pharmaceutical composition of claim 13 comprising the YFV-17D prME gene cassette set out in SEQ ID NO: 3.

19. The pharmaceutical composition of claim 13 wherein the prME gene cassette is inserted in the MVA in the deletion I region, the deletion II region, the deletion III region, the deletion IV region, the thymidine kinase locus, the D4/5 intergenic region, or the HA locus.

20-24. (canceled)

25. A method of inducing an immune response to YFV in an individual comprising administering to the individual a pharmaceutical composition comprising a recombinant, modified vaccinia virus Ankara (MVA) comprising a gene cassette encoding a yellow fever virus (YFV) prME polypeptide.

26. The method of claim 25 wherein the gene cassette encodes a YFV prME amino acid sequence set out in SEQ ID NO: 2, SEQ ID NO: 5, SEQ ID NO: 6, SEQ ID NO: 7, SEQ ID NO: 8, SEQ ID NO: 9, or SEQ ID NO: 10.

27. The method of claim 25 wherein expression of the YFV prME polypeptide from the gene cassette is under the control of an mH5 promoter or selP promoter.

28. (canceled)

29. The method of claim 25 of inducing an immune response to YFV in an individual comprising administering to the individual a pharmaceutical composition comprising the YFV-17D prME gene cassette set out in SEQ ID NO: 1.

30. The method of claim 25 of inducing an immune response to YFV in an individual comprising administering to the individual a pharmaceutical composition comprising the YFV-17D prME gene cassette set out in SEQ ID NO: 3.

31. The method of claim 25 wherein the prME gene cassette is inserted in the MVA in the deletion I region, the deletion II region, the deletion III region, the deletion IV region, the thymidine kinase locus, the D4/5 intergenic region, or the HA locus.

32-36. (canceled)

37. The method of claim 25, wherein the pharmaceutical composition is administered as a single dose.

38-74. (canceled)
